SEQUENCE LISTING (24 sequences)

SEQ ID NO: 1 - protein, 37 aa, Pseudopleuronectes americanus
DTASDAAAAAALTAANAKAAAELTAANAAAAAAATAR

SEQ ID NO: 2 - protein, 37 aa, Pseudopleuronectes americanus
DTASDAAAAAALTAANAKAAAKLTADNAAAAAAATAR

SEQ ID NO: 3 - protein, 49 aa, Limanda ferruginea
DTASDAAAAAAATAAAAAKAAADTAAAAAKAAADTAAAAAEAAAATARG

SEQ ID NO: 4 - protein, 33 aa, Myoxocephalus scorpius
MNAPARAAAKTAADALAAAKKTAADAAAAAAAA

SEQ ID NO: 5 - protein, 45 aa, Myoxocephalus scorpius
MNGETPAQKAARLAAAAALAAKTAADAAAKAAAKAAAIAAAAASA

SEQ ID NO: 6 - protein, 33 aa, Myoxocephalus aenaeus
MDAPAIAAAKTAADALAAAKKTAADAAAAAAKP

SEQ ID NO: 7 - protein, 33 aa, Myoxocephalus scorpiodes
MDAPARAAAKTAADALAAANKTAADAAAAAAAA

SEQ ID NO: 8 - protein, 32 aa, Myoxocephalus scorpiodes
MDGETPAQKAARKAAAAAALAKTAADAAAAAA

SEQ ID NO: 9 - protein, 166 aa, Hemitripterus americanus
TTRMLTVSLLVCAMMALTQANDDKILKGTATEAGPVSQRAPPNCPAGWGPLGDRCIYYETTAMTWALAETNCMKLGGHLASIHSQEGHSFIQTLNAGVVWIGGSACLQAGAWTWSDGTPMNFRSWCSTKPDDVLAACCMQMTAAADQCWDDLPCPASHKSVCAMTF

SEQ ID NO: 10 - protein, 20 aa, Lycodes polaris
NKASVVANQLIPINTALTLV

SEQ ID NO: 11 - protein, 20 aa, Rhigophila dearborni
NKASVVANQLIPINTALTLI

SEQ ID NO: 12 - protein, 20 aa, Macrozoarces americanus
ASQSVVATQLIPINTALTPA

SEQ ID NO: 13 - protein, 20 aa, Lycodes polaris
MMRAEVVTPAGIPAEDIPRL

SEQ ID NO: 14 - protein, 20 aa, Rhigophila dearborni
MMKAEVVTPMGIPAEDIPRI

SEQ ID NO: 15 - protein, 20 aa, Macrozoarces americanus
MMEGKVTNPIGIPFAEMSQI

SEQ ID NO: 16 - protein, 16 aa, Macrozoarces americanus
KVTNPIGIPFAEMSQI

SEQ ID NO: 17 - protein, 20 aa, Lycodes polaris
VGLQVNRAVLIGTTLMPDMV

SEQ ID NO: 18 - protein, 20 aa, Rhigophila dearborni
IGMQVNRAVPLGTTLMPDMV

SEQ ID NO: 19 - protein, 20 aa, Macrozoarces americanus
VGKQVNTPVAKGQTLMPNMV

SEQ ID NO: 20 - protein, 20 aa, Macrozoarces americanus
VGKQVNTPVAKGQTIMPNMV

SEQ ID NO: 21 - protein, 6 aa, Lycodes polaris
KGYAPQ

SEQ ID NO: 22 - protein, 4 aa, Rhigophila dearborni
KNYE

SEQ ID NO: 23 - protein, 7 aa, Macrozoarces americanus
KTYVAGK

SEQ ID NO: 24 - protein, 7 aa, Macrozoarces americanus
KTYAAGK

(19) United States
(12) Patent Application Publication
(10) Pub. No.: US 2006/0008440 A1
(43) Pub. Date: Jan. 12, 2006

(54) Cosmetic or dermatological preparations having a content of anti-freezing proteins and/or anti-freezing glycoproteins

(75) Inventors: Thomas Blatt, Hamburg (DE); Christopher Mummert, Bienenbuettel (DE); Claudia Mundt, Bremen (DE); Franz Staeb, Echem (DE)

Correspondence Address: GREENBLUM & BERNSTEIN, P.L.C., 1950 ROLAND CLARKE PLACE, RESTON, VA, 20191, US

(73) Assignee: BEIERSDORF AG, Hamburg (DE)

(21) Appl. No.: 11/071,259

(22) Filed: Mar. 4, 2005

Related U.S. Application Data
(63) Continuation of application No. PCT/EP02/10044, filed on Sep. 7, 2002.

(51) Int. Cl. A61K 8/64; C07K 14/47

(52) U.S. Cl. 424/70.14; 530/350

(57) ABSTRACT

A cosmetic or dermatological preparation that comprises one or more anti-freezing proteins and/or anti-freezing glycoproteins.

### CROSS-REFERENCE TO RELATED APPLICATIONS

The present application is a continuation of International Application No. PCT/EP2002/010044, filed Sep. 7, 2002, the entire disclosure whereof is expressly incorporated by reference herein.

### BACKGROUND OF THE INVENTION

1. Field of the Invention

The present invention relates to cosmetic or dermatological preparations with a content of one or more “anti-freezing proteins” (also referred to below as AFPs) and/or one or more “anti-freezing glycoproteins” (also referred to below as AFGPs).

2. Discussion of Background Information

In particular, the invention relates to cosmetic or dermatological preparations for the prophylaxis of degenerative skin phenomena and treatment of cold-related structural and cellular damage in the skin which with distinct climate and weather-induced drops in temperature, through the loss of the temperature optima of cellular enzymes, cause changes in the cell physiology in the cell and in the extracellular space, further for the prophylaxis and/or treatment of sensitive and dry skin, against itching and for the protection of the skin, the lips and the mucous membranes of the mouth and nose and the integumentary appendages against harmful environmental influences.

In a further embodiment, the present invention relates to cosmetic preparations with an effective protection against harmful environmental influences, such as cold, heat, considerable fluctuations in temperature, UV light, smog, oxidation processes in the skin, as well as for the additional protection of cosmetic preparations themselves or for additional protection of the constituents of cosmetic preparations against harmful oxidation processes and influences through unfavorable temperature changes.

The terms cosmetic skin care and skin protection primarily mean the strengthening, maintaining or rebuilding of the skin's natural function as a barrier against environmental influences (e.g. dirt, chemicals, microorganisms) and against the loss of endogenous substances (e.g. water, natural fats, electrolytes).

Impairment of this function may lead to metabolic disturbances in the skin and, e.g., to increased resorption of toxic or allergenic substances or attack by microorganisms, leading to toxic or allergic skin reactions.

Another aim of skin care is to compensate for the loss by the skin of lipids and water caused by daily washing. This is particularly important when the natural regeneration ability is insufficient. Furthermore, skin care products should protect against environmental influences, in particular against sun and wind, and delay skin aging.

Chronological skin aging is caused, for example, by endogenous genetically determined factors. The following structural damage and functional disorders, which can also fall under the term “senile xerosis,” result, for example, in the epidermis and dermis as a result of aging:
a) dryness, roughness and formation of dryness wrinkles,
b) itching and
c) reduced regreasing by sebacious glands (e.g. after washing).

Exogenous factors, such as unphysiological temperatures, pronounced temperature fluctuations, wind, UV light and chemical noxae, can have a cumulative effect. In the epidermis and dermis, for example, the following structural damage and functional disorders appear in the skin as a result of exogenous factors:
d) increased susceptibility to chemical and mechanical stress (e.g., cracking, flaccidity, redness and feeling of tightness of the skin).

Products for the care and protection of sensitive, itching and/or dry skin or products for the treatment or prophylaxis of signs of skin aging are known per se. However, their effectiveness is limited.

The harmful effect of the ultraviolet part of solar radiation on the skin is generally known. Whereas rays with a wavelength of less than 290 nm (the so-called UVC region) are absorbed by the ozone layer in the earth's atmosphere, rays in the range between 290 nm and 320 nm, the so-called UVB region, cause erythema, simple sunburn or even burns of greater or lesser severity.

A maximum erythema activity of sunlight occurs in the relatively narrow range around 308 nm.

Numerous compounds are known for protecting against UVB radiation; these are derivatives of 3-benzylidene camphor, 4-aminobenzoic acid, cinnamic acid, salicylic acid, benzophenone and also 2-phenylbenzimidazole.

It is also important to have available filter substances for the range between about 320 nm and about 400 nm, the so-called UVA region, since the corresponding radiation can cause reactions in cases of photosensitive skin. It has been found that UVA radiation leads to damage of the elastic and collagenous fibers of connective tissue, which leads to premature aging of the skin, and is regarded as a cause of numerous phototoxic and photoallergic reactions. The harmful effect of UVB radiation can be intensified by UVA radiation.

To protect against rays of the UVA region, certain derivatives of dibenzoylmethane are used, the photostability of which is inadequate (Int. J. Cosm. Science 10, 53 (1988)).

The UV radiation can, however, also lead to photochemical reactions, in which case the photochemical reaction products intervene in the skin metabolism.

Such photochemical reaction products are predominantly free-radical compounds, for example hydroxyl radicals. Undefined free-radical photoproducts which form in the skin itself can also display uncontrolled secondary reactions because of their high reactivity. However, singlet oxygen, a non-free-radical excited state of the oxygen molecule, can also be formed during UV irradiation, as can short-lived epoxides and many others. Singlet oxygen, for example, differs from normal triplet oxygen (free-radical ground state) by virtue of its increased reactivity. However, excited, reactive (free-radical) triplet states of the oxygen molecule also exist.

UV radiation is also a type of ionizing radiation. There is therefore the risk that ionic species will also form during UV exposure, which then for their part are able to intervene oxidatively in the biochemical processes.

In order to prevent these reactions, additional antioxidants and/or free-radical scavengers can be incorporated into the cosmetic or dermatological formulations.

It has already been proposed to use vitamin E, a substance with known antioxidant action, in sunscreen formulations, although, here too, the effect achieved falls a long way short of expectations.

Antioxidants are mainly used as substances which protect against the deterioration of the preparations in which they are present. Nevertheless, it is known that undesired oxidation processes may occur in human or animal skin as well.

The article “Skin Diseases Associated with Oxidative Injury” in “Oxidative Stress in Dermatology”, p. 323 ff. (Marcel Decker Inc., New York, Basel, Hong Kong, Editor: Jurgen Fuchs, Frankfurt, and Lester Packer, Berkeley/California) discusses oxidative skin damage and its more detailed causes.

Also for the reason of preventing such reactions, antioxidants and/or free-radical scavengers can be additionally incorporated into cosmetic or dermatological formulations.

A number of antioxidants and free-radical scavengers are known. For example U.S. Pat. Nos. 4,144,325 and 4,248,861, and numerous other documents have already proposed the use of vitamin E, a substance with known antioxidative action, in sunscreen formulations, although here too the effect achieved falls a long way short of the desired effect.

In view of the foregoing, it is desirable to have available cosmetic, dermatological and pharmaceutical active substances and preparations as well as sunscreen preparations for the protection and treatment of photo-sensitive and temperature-sensitive skin, in particular of areas of the skin that are intensively exposed to environmental influences, such as, e.g., areas of the face, the lips, the nose, the forehead, the eyes, the head, the décolleté and the hands and arms.

It also is desirable to find ways of avoiding the disadvantages of the prior art. In particular, the effect of repairing or preventing damage caused by environmental noxae should be permanent, sustained and without the risk of side effects.

### SUMMARY OF THE INVENTION

It has surprisingly been found that the use of substances selected from anti-freezing proteins and anti-freezing glycoproteins in cosmetic or dermatological preparations for the treatment, care and prophylaxis in the case of signs of aged skin, with sensitive, dry and temperature-sensitive skin and/or for the treatment and prophylaxis of the symptoms of a negative change of the physiological homeostasis of the healthy and young skin remedy the disadvantages of the prior art.

Accordingly, the present invention provides a cosmetic or dermatological preparation which comprises one or more proteins which are selected from anti-freezing proteins and anti-freezing glycoproteins.

In one aspect, the preparation may comprise at least one anti-freezing protein. In another aspect, it may comprise at least one anti-freezing glycoprotein.

In yet another aspect of the preparation, the one or more proteins may be present in a concentration of from 0.0001% to 50% by weight, based on the total weight of the preparation, e.g., in a concentration of from 0.001% to 50% by weight, of from 0.1% to 10% by weight, or from 0.1% to 1% by weight.

In a still further aspect, the at least one anti-freezing protein may comprise at least one protein selected from types AFP 1, AFP 2, AFP 3 and AFP 4, for example, at least one protein of type AFP 1 that is synthesized by pseudopluronectes americanus, myoxocephalus scorpius, myoxocephalus aenaeus and/or myoxocephalus scorpiodes, at least one protein of type AFP 2 that is synthesized by hemitripterus americanus, osmerus mordax and/or clupea harengus harengus, at least one protein of type AFP 3 that is synthesized by macrozoarces americanus, rhigophila dearbomi lycodes polaris and/or the “wolf fish” anarhichas lupus, and/or at least one protein of type AFP 4 that is synthesized by myoxocephalus octodecimspinosis. In another aspect, the at least one anti-freezing glycoprotein may comprise at least one protein that is synthesized by trematomas borgrevinki, dissostichus mawsoni, boreogadus saida and/or gadus morhua.

In another aspect of the preparation of the present invention, at least a part of the one or more proteins may be encapsulated.

The present invention also provides a cosmetic or dermatological preparation which comprises one or more proteins which are selected from anti-freezing proteins and anti-freezing glycoproteins that are synthesized by at least one of pseudopluronectes americanus, myoxocephalus scorpius, myoxocephalus aenaeus, myoxocephalus scorpiodes, hemitripterus americanus, osmerus mordax, clupea harengus harengus, macrozoarces americanus, rhigophila dearborni, lycodes polaris, anarhichas lupus, myoxocephalus octodecimspinosis, trematomas borgrevinki, dissostichus mawsoni, boreogadus saida and gadus morhua.

In one aspect, the one or more proteins may be present in a concentration of from 0.001% to 50% by weight, based on the total weight of the preparation, e.g., in a concentration of from 0.1% to 10% by weight.

The present invention also provides a cosmetic or dermatological product which is an o/w cream, a w/o cream, a w/o/w cream, an o cream, a w/o emulsion, a hydrodispersion, a gel cream, a w/o stick or an o stick, and which comprises the preparation of the present invention, including the various aspects thereof.

The present invention also provides a method for the treatment or prevention of undesirable skin conditions. The method comprises applying one or more proteins to at least parts of the skin, which proteins are selected from anti-freezing proteins and anti-freezing glycoproteins.

In one aspect, the undesirable skin conditions may include skin inflammation, pigmentation disorders, symptoms of extrinsic and intrinsic skin aging and/or skin damage caused by UV radiation.

The term “anti-freezing proteins” is used by those of skill in the art to describe proteins that enable an organism, even under extreme temperature conditions, to keep important cell structures functionally active. In view of their function, “anti-freezing proteins” in this sense also represent “frost-protection compounds” on a cellular level.

The best studied anti-freezing glycoproteins come from Arctic fish, such as, e.g., trematomas borgrevinki and dissostichus mawsoni, and from Nordic fish, such as, e.g., boreogadus saida and gadus morhua. The best tested type 1 anti-freezing proteins come from the pseudopleuronectes americanus, myoxocephalus scorpius, myoxocephalus aenaeus, myoxocephalus scorpiodes, of the type 2 from the hemitripterus americanus, osmerus mordax and clupea harengus harengus, of the type 3 from macrozoarces americanus, rhigophila dearborni, lycodes polaris and the “wolf fish” anarhichas lupus and of type 4 from myoxocephalus octodecimspinosis.

The AFPs are represented, inter alia, by the following amino acid sequences (source: Ananthanarayanan VS: Antifreeze proteins: structural diversity and mechanism of action. Life Chem Rep 7: 1-32, 1989).

a) Type I AFP

Pseudopleuronectes americanus

| | |
|---|---|
| | 1 10 20 30 |
| HPLC-6 | DTASDAAAAAALTAANAKAAAELTAANAAAAAAATAR |
| hplc-8 | DTASDAAAAAALTAANAKAAAKLTADNAAAAAAATAR |

Limanda ferruginea

1 10 20 30 40
DTASDAAAAAAATAAAAAKAAADTAAAAAKAAADTAAAAAE
AAAATARG

Myoxocephalus scorpius

| | |
|---|---|
| | 1 5 10 20 30 |
| SS-3 | MN APARAAAKTAADALAAAKKTAADAAAAAAAA |
| | 1 10 20 30 40 |
| SS-8 | MNGETPAQKAARLAAAAALAAKTAADAAAKAAAKAAAIAAA |
| | AASA |

Myoxocephalus aeneus

| | |
|---|---|
| | 1 5 10 20 30 |
| GS-5 | MD APAIAAAKTAADALAAAKKTAADAAAAAAKP |

Myoxocephalus scorpiodes

| | |
|---|---|
| | 1 5 10 20 30 |
| AS-1 | MD APARAAAKTAADALAAANKTAADAAAAAAAA |
| | 1 10 20 30 |
| AS-3 | MDGETPAQKAARKAAAAAALAKTAADAAAAAA |

b) Type II AFP from Hemitripterus americanus

Thr Thr Arg Met Leu Thr Val Ser Leu Leu Val Cys Ala Met Met Ala Leu Thr Gln Ala
1 10 20
Asn Asp Asp Lys Ile Leu Lys Gly Thr Ala Thr Glu Ala Gly Pro Val Ser Gln Arg Ala
21 30 40
Pro Pro Asn Cys Pro Ala Gly Trp Gly Pro Leu Gly Asp Arg Cys Ile Tyr Tyr Glu Thr
41 50 60
Thr Ala Met Thr Trp Ala Leu Ala Glu Thr Asn Cys Met Lys Leu Gly Gly His Leu Ala
61 70 80
Ser Ile His Ser Gln Glu Gly His Ser Phe Ile Gln Thr Leu Asn Ala Gly Val Val Trp
81 90 100
Ile Gly Gly Ser Ala Cys Leu Gln Ala Gly Ala Trp Thr Trp Ser Asp Gly Thr Pro Met
101 110 120
Asn Phe Arg Ser Trp Cys Ser Thr Lys Pro Asp Asp Val Leu Ala Ala Cys Cys Met Gln
121 130 140
Met Thr Ala Ala Ala Asp Gln Cys Trp Asp Asp Leu Pro Cys Pro Ala Ser His Lys Ser
141 150 160
Val Cys Ala Met Thr Phe
161

c) Type III AFP

| | |
|---|---|
| | 1 10 20 |
| LP^{b} | Asn Lys Ala Ser Val Val Ala Asn Gln Leu Ile Pro Ile Asn Thr Ala Leu Thr Leu Val |
| RD^{c} | Asn Lys Ala Ser Val Val Ala Asn Gln Leu Ile Pro Ile Asn Thr Ala Leu Thr Leu Ile |
| SPI-C^{d} | Ala Ser Gln Ser Val Val Ala Thr Gln Leu Ile Pro Ile Asn Thr Ala Leu Thr Pro Ala |
| | 21 30 40 |
| LP | Met Met Arg Ala Glu Val Val Thr Pro Ala Gly Ile Pro Ala Glu Asp Ile Pro Arg Leu |
| RD | Met Met Lys Ala Glu Val Val Thr Pro Met Gly Ile Pro Ala Glu Asp Ile Pro Arg Ile |
| SP1-C | Met Met Glu Gly Lys Val Thr Asn Pro Ile Gly Ile Pro Phe Ala Glu Met Ser Gln Ile |
| SP1-A^{d} | Lys Val Thr Asn Pro Ile Gly lIe Pro Phe Ala Glu Met Ser Gln Ile |
| | 41 50 60 |
| LP | Val Gly Leu Gln Val Asn Arg Ala Val Leu Ile Gly Thr Thr Leu Met Pro Asp Met Val |
| RD | Ile Gly Met Gln Val Asn Arg Ala Val Pro Leu Gly Thr Thr Leu Met Pro Asp Met Val |
| SP1-C | Val Gly Lys Gln Val Asn Thr Pro Val Ala Lys Gly Gln Thr Leu Met Pro Asn Met Val |
| SP1-A | Val Gly Lys Gln Val Asn Thr Pro Val Ala Lys Gly Gln Thr Ile Met Pro Asn Met Val |
| | 61 |
| LP | Lys Gly Tyr Ala Pro Gln |
| RD | Lys Asn Tyr Glu |
| SP1-C | Lys Thr Tyr Val Ala Gly Lys |
| AP1-A | Lys Thr Tyr Ala Ala Gly Lys |

In the above examples of amino acid sequences of the various AFP types, amino acids are given in a one-letter code in a), and in a three-letter code in b) and c); abbreviations used: LP=Lycodes polaris, RD=Rhigophila dearbomi, SP1-A and SP1-C=sequences of macrozoarces americanus.

Cosmetic or dermatological preparations according to the present invention containing AFP and/or AFGP are in every respect most satisfactory preparations.

It was not foreseeable for those of skill in the art that the preparations according to the present invention
protect better against structural and cellular damage in the skin due to cold
better maintain or restore the barrier properties of the skin
better combat drying out of the skin
act better against dyschromia
act better against inflammatory skin conditions
act better against skin aging, and
better protect the skin against environmental influences
   than the preparations of the prior art.

The use of AFP and/or AFGP or cosmetic or topical dermatological preparations with an effective content of AFP and/or AFGP surprisingly renders possible an effective treatment, but also a prophylaxis of
structural and cellular damage in the skin due to cold, which damage with distinct climate- and weather-induced drops in temperature cause changes in the cell physiology in the cell and in the extracellular space through loss of the temperature optima of cellular enzymes.
skin damage, skin redness and tight feeling of the skin and increased sensory sensitivities, induced, e.g., by cold, wind and/or UV light,
temperature-sensitive skin,
negative changes in the skin, the lips and the mucous membranes in the nose and mouth area and the integumentary appendage caused by environmental stress (caused by temperature changes and UV light, smoking, smog, reactive oxygen species, free radicals).

The use of AFP and/or AFGP or cosmetic or topical dermatological preparations with an effective content of AFP and/or AFGP is, surprisingly, an effective treatment as well as a prophylaxis
of deficient, sensitive or hypoactive skin conditions or deficient, sensitive or hypoactive conditions of integumentary appendages
of signs of premature aging of the skin (e.g., wrinkles, senile keratoses, telangiectases) and/or of the integumentary appendages,
of environmentally induced (smoking, smog, reactive oxygen species, free radicals) and in particular light-induced negative changes in the skin and the integumentary appendages,
of light-induced skin damage,
of pigmentation disorders,
of sensitive, irritated and itchy skin,
of dry skin conditions and disorders of the horny layer barrier,
of hair loss and for improved hair growth,
signs of skin aging, such as, e.g., wrinkles and reduced skin regeneration,
of inflammatory skin conditions, and atopic eczema, seborrhoeic eczema, polymorphous photodermatosis, psoriasis, vitiligo,
to sooth sensitive or irritated skin,
to stimulate the synthesis of collagen, hyaluronic acid and elastin,
changes of the normal hyaluronic acid and glycosaminoglycan content of healthy skin,
to stimulate the ceramide synthesis of the skin
to stimulate intracellular DNA synthesis, in particular in cases of deficient or hypoactive skin conditions,
to increase cell renewal and regeneration of the skin,
to increase the skin's own protective and repair mechanisms (for example, for dysfunctional enzymes, DNA, lipids, proteins),
reduction in cell-cell communication
deficient, sensitive or hypoactive skin conditions or deficient, sensitive or hypoactive conditions of skin appendages,
a change in the ceramide, lipid and energy metabolism of healthy skin,
changes in lipid and protein peroxidation,
a change in the physiological transepidermal water loss,
a reduction in skin hydration, normal osmoregulation and decrease in the moisture content of the skin,
change in the natural moisturizing factor content,
DNA damage and reduction in endogenous DNA repair mechanisms,
activation of metalloproteinases and/or other proteases or inhibition of the corresponding endogenous inhibitors of these enzymes,
deviations from the normal post-translational modifications of connective tissue constituents of healthy skin,
dandruff formation in the hair and hair region,
brittleness of the skin, loss of elasticity and skin fatigue,
increase in the normal keratinocyte proliferation,
reduction of the natural regeneration and structure of the skin and hair
for pre- and post-treatment in cases of topical application of laser and abrasive treatments, which serve, for example, to reduce skin wrinkles and scars, to counteract the resulting skin irritations and to promote the regeneration processes in the damaged skin.

Accordingly, the use of AFPs and/or AFGPs for the prophylaxis and treatment of inflammatory skin conditions—also atopical eczema—and/or for skin protection in the case of skin predisposed to be sensitive and dry is also in accordance with the invention.

Accordingly, the use of cosmetic or dermatological preparations for the production of cosmetic or dermatological preparations for the treatment and/or prophylaxis of pigmentation disorders is also in accordance with the invention.

Accordingly, the use of preparations for the production of cosmetic or dermatological preparations for the treatment and/or prophylaxis of the symptoms of intrinsic and/or extrinsic skin aging and for the treatment and prophylaxis of harmful effects of ultraviolet radiation on the skin is also according to the invention.

Accordingly, the use of AFPs and/or AFGPs for the production of cosmetic or dermatological preparations for increasing ceramide biosynthesis is also according to the invention.

Accordingly, the use of AFPs and/or AFGPs for the production of cosmetic or dermatological preparations for strengthening the barrier function of the skin is also in accordance with the invention.

Cosmetic or dermatological preparations according to the present invention preferably contain from 0.0001% to 50% by weight, particularly preferably from 0.01% to 10% by weight, of the cited AFPs and/or AFGPs or a combination of two or more of the cited AFPs and/or AFGPs, based on the total weight of the preparations.

According to the present invention, it is in particular extremely advantageous to use AFPs and/or AFGPs or cosmetic or topical dermatological preparations with an effective content of AFPs and/or AFGPs for the cosmetic or dermatological treatment or prophylaxis of undesirable skin conditions.

According to the present invention, customary antioxidants can be used in the preparations that contain the active substance combinations according to the present invention.

Advantageously, the antioxidants are selected from the group of amino acids (for example, glycine, histidine, tyrosine, tryptophan, β-alanine) and derivatives thereof, imidazoles (for example urocanic acid) and derivatives thereof, peptides, such as D,L-carnosine, D-carnosine, L-carnosine and derivatives thereof (for example, anserine), carotenoids, carotenes (for example, α-carotene, β-carotene, lycopene) and derivatives thereof, lipoic acid and derivatives thereof (for example, dihydrolipoic acid), aurothioglucose, propylthiouracil and other thiols (for example, thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (for example, buthionine sulfoximines, homocysteine sulfoximine, buthionine sulfones, penta-, hexa- and heptathionine sulfoximine) in very low tolerated doses (for example pmol to μmol/kg), and furthermore (metal) chelating agents (for example, α-hydroxy fatty acids, palmitic acid, phytic acid, lactoferrin), α-hydroxy acids (for example, citric acid, lactic acid, malic acid), humic acid, bile acid, bile extracts, bilirubin, biliverdin, EDTA, EGTA and derivatives thereof, unsaturated fatty acids and derivatives thereof (for example γ-linolenic acid, linoleic acid, oleic acid), folic acid and derivatives thereof, alanine diacetic acid, flavonoids, polyphenols, catechols, vitamin C and derivatives thereof (e.g., ascorbyl palmitate, Mg-ascorbyl phosphate, ascorbyl acetate), tocopherols and derivatives thereof (for example, vitamin E acetate), and coniferyl benzoate of benzoin resin, rutinic acid and derivatives thereof, ferulic acid and derivatives thereof, butylated hydroxytoluene, butylated hydroxyanisole, nordihydroguaiacic acid, nordihydroguaiaretic acid, trihydroxybutyrophenone, uric acid and derivatives thereof, mannose and derivatives thereof, zinc and derivatives thereof (for example, ZnO, ZnSO₄), selenium and derivatives thereof (for example selenomethionine), stilbenes and derivatives thereof (for example stilbene oxide, trans-stilbene oxide) and the derivatives of these active ingredients mentioned which are suitable according to the invention (salts, esters, ethers, sugars, nucleotides, nucleosides, peptides and lipids).

The amount of the antioxidants (one or more compounds) in the preparations is preferably from 0.001% to 30% by weight, particularly preferably from 0.05% to 20% by weight, particularly preferred from 1% to 10% by weight, based on the total weight of the preparation.

In addition, it may be advantageous to encapsulate the active ingredients according to the invention, as so-called solid lipid nanoparts using molten waxes, which may be chosen, inter alia, but not exclusively, from ester waxes, triglyceride waxes or hydrocarbon waxes. In addition, it may be advantageous to encapsulate the active ingredients according to the invention in polymers, e.g., in particles based on highly crosslinked polymethacrylates and/or cellulose triacetates and/or as core/shell particles with a shell made of poly(oxymethylurea), nylon, polyamides, polyurethane, polyester, gelatin and polyolefins.

The prophylaxis or the cosmetic or dermatological treatment with the active ingredient used according to the invention or with the cosmetic or topical dermatological preparations having an effective content of active ingredient used according to the invention may be carried out in the usual manner, by applying the active ingredient used according to the invention or the cosmetic or topical dermatological preparations having an effective content of active ingredient used according to the invention to the affected areas of the skin.

The active ingredient used according to the invention can advantageously be incorporated into customary cosmetic and dermatological preparations which may assume various forms. Thus, they may, for example, be a solution, an emulsion of the water-in-oil (W/O) type or of the oil-in-water (O/W) type, or a multiple emulsion, for example of the water-in-oil-in-water (W/O/W) type or oil-in-water-in-oil (O/W/O) type, a hydrodispersion or lipodispersion, a gel, a Pickering emulsion, a solid stick or an aerosol.

Emulsions according to the invention for the purposes of the present invention, e.g., in the form of a cream, a lotion, a cosmetic milk, and a stick, are advantageous and may comprise, for example, fats, oils, waxes and/or other fatty substances, and water and one or more emulsifiers as are customarily used for this type of formulation.

It is also possible and advantageous for the purposes of the present invention to incorporate the active ingredient used in accordance with the present invention into aqueous systems or surfactant preparations for cleansing and treating the skin and the hair.

One of skill in the art is, of course, aware that demanding cosmetic compositions are almost inconceivable without the customary auxiliaries and additives. Examples thereof include builders, fillers, perfume, dyes, emulsifiers, additional active ingredients, such as vitamins or proteins, light protection agents, stabilizers, insect repellents, alcohol, water, salts, and antimicrobially, proteolytically or keratolytically active substances, etc.

Corresponding requirements apply mutatis mutandis to the formulation of medicinal preparations.

Medicinal topical compositions for the purposes of the present invention generally comprise one or more medicaments in an effective concentration. For the sake of simplicity, for a clear distinction between cosmetic and medicinal application and corresponding products, reference is made to the legal provisions of the Federal Republic of Germany (e.g., Cosmetics Directive, Foods and Drugs Act).

In this connection, it is likewise advantageous to add the active ingredient used according to the invention as an additive to preparations which already comprise other active ingredients for other purposes.

Accordingly, for the purposes of the present invention, cosmetic or topical dermatological compositions can, depending on their formulation, be used, for example, as skin protection cream, cleansing milk, sunscreen lotion, nourishing cream, day or night cream, lip care stick, nasal spray, etc. In some instances it is possible and advantageous to use the compositions according to the invention as bases for pharmaceutical formulations.

It is also advantageous for the purposes of the present invention to provide cosmetic and dermatological preparations whose main purpose is not protection against sunlight, but which nevertheless have a content of UV protection substances. Thus, for example, UVA and/or UVB filter substances are usually incorporated into day creams or makeup products. Also UV protection substances, likewise antioxidants and, if desired, preservatives, provide an effective protection of the preparations against deterioration. Furthermore, cosmetic and dermatological preparations are favorable which are in the form of a sunscreen.

Accordingly, the preparations according to the present invention, in addition to one or more active ingredient combinations according to the invention, preferably additionally comprise at least one further UVA filter substance and/or UVB filter substance. The formulations can, although this is not necessary, optionally also comprise one or more organic and/or inorganic pigments as UV filter substances, which can be present in the aqueous phase and/or the oil phase.

Preferred inorganic pigments are metal oxides and/or other metal compounds which are insoluble or sparingly soluble in water, in particular the oxides of titanium (TiO₂), zinc (ZnO), iron (e.g., Fe₂O₃), zirconium (ZrO₂), silicon (SiO₂), manganese (e.g. MnO), aluminum (Al₂O₃), cerium (e.g., Ce₂O₃), mixed oxides of the corresponding metals, and mixtures of such oxides.

According to the invention such pigments can advantageously be surface-treated (“coated”) whereby, e.g., an amphiphilic or hydrophobic character of these pigments is to be formed or retained. This surface treatment can comprise providing the pigments with a thin hydrophobic layer by methods known per se.

According to the invention, e.g., titanium dioxide pigments are advantageous that are coated with octylsilanol. Suitable titanium dioxide particles are available under the trade name T805 from Degussa. Furthermore, TiO₂ pigments coated with aluminum stearate are particularly advantageous, e.g., those available under the trade name MT 100 T from TAYCA.

A further advantageous coating of the inorganic pigments comprises dimethylpolysiloxane (also: dimethicone), a mixture of completely methylated, linear siloxane polymers which are terminally blocked with trimethylsiloxy units. For the purposes of the present invention, particularly advantageous pigments are zinc oxide pigments which are coated in this way.

Also advantageous is a coating of the inorganic pigments with a mixture of dimethylpolysiloxane, in particular dimethylpolysiloxane having an average chain length of from 200 to 350 dimethylsiloxane units, and silica gel, which is also referred to as simethicone. It is particularly advantageous if the inorganic pigments have been additionally coated with aluminium hydroxide or hydrated aluminium oxide (also alumina, CAS No.: 1333-84-2). Particularly advantageous are titanium dioxides which have been coated with simethicone and alumina, it being possible for the coating to also comprise water. One example thereof is the titanium dioxide available under the trade name Eusolex T2000 from Merck.

An advantageous organic pigment for the purposes of the present invention includes 2,2′-methylenebis-(6-(2H-benzotriazol-2-yl)-4-(1,1,3,3-tetramethylbutyl)phenol) [INCI: Bisoctyltriazole], which is obtainable from CIBA Chemikalien GmbH under the trade name Tinosorb® M.

Advantageously, preparations according to the invention contain substances that absorb UV radiation in the UVA and/or the UVB range, whereby the total amount of the filter substances is, e.g., from 0.1% by weight to 30% by weight, preferably from 0.5 to 20% by weight, in particular from 1.0 to 15% by weight, based on the total weight of the preparations, in order to provide cosmetic preparations that protect the hair or the skin against the entire range of ultraviolet radiation. They can also be used as sunscreen for the hair or the skin.

Further advantageous UVA filter substances for the purposes of the present invention include dibenzoylmethane derivatives, in particular 4-(tert-butyl)-4′-methoxydibenzoylmethane (CAS No. 70356-09-1), which is sold by Givaudan under the trademark Parsol® 1789 and by Merck under the trade name Eusolex® 9020.

Advantageous further UVA filter substances include phenylene-1,4-bis-(2-benzimidazyl)-3,3′,5,5′-tetrasulfonic acid and its salts, particularly the corresponding sodium, potassium or triethanolammonium salts, in particular the phenylene-1,4-bis-(2-benzimidazyl)-3,3′,5,5′-tetrasulfonic acid bis-sodium salt with the INCI name Bisimidazylate, which is available, for example, under the trade name Neo Heliopan AP from Haarmann & Reimer.

Also advantageous are 1,4-di(2-oxo-10-sulfo-3-bornylidenemethyl)benzene and salts thereof (particularly the corresponding 10-sulfato compounds, in particular the corresponding sodium, potassium or triethanolammonium salt), which is also referred to as benzene-1,4-di(2-oxo-3-bornylidenemethyl-10-sulfonic acid).

Advantageous UV filter substances for the purposes of the present invention are also so-called broadband filters, i.e., filter substances which absorb both UVA and UVB radiation.

Advantageous broadband filters or UVB filter substances include, for example, bis-resorcinyltriazine derivatives. Particularly preferred are 2,4-bis{[4-(2-ethylhexyloxy)-2-hydroxylphenyl}-6-(4-methoxyphenyl)-1,3,5-triazine (INCI: Aniso Triazine), which is available under the trade name Tinosorb® S from CIBA-Chemikalien GmbH.

Particularly advantageous preparations for the purposes of the present invention that are characterized by a high or very high UVA protection preferably contain several UVA and/or broadband filters, in particular dibenzoylmethane derivatives [e.g., 4-(tert.butyl)-4′-methoxydibenzoylmethane], benzotriazole derivatives [e.g., 2,2′methylene-bis-(6-(2H-benzotriazol-2-yl)-4-(1,1,3,3-tetramethylbutyl)-phenol], phenylene-1,4-bis-(2-benzimidazyl)-3,3′,5,5′-tetrasulfonic acid and/or salts thereof, 1,4-di(2-oxo-10-sulfo-3-bornylidenemethyl)-benzene and/or salts thereof and/or 2,4-bis-{[4-(2-ethylhexyloxy)-2-hydroxy]-phenyl}-6-(4-methoxyphenyl)-1,3,5-triazine, individually or in any combinations with one another.

Other UV filter substances that have the structural formula

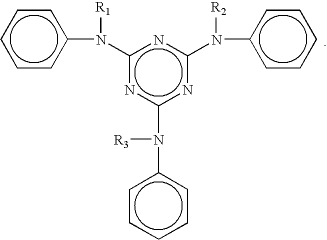

Are also advantageous UV filter substances for the purposes of the present invention, for example the s-triazine derivatives described in European patent application EP 570 838 A1, the chemical structure of which is given by the generic formula 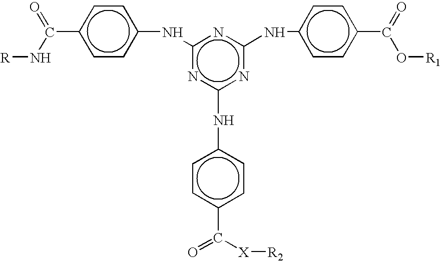
where
R is a branched or unbranched C₁-C₁₈-alkyl radical, a C₅-C₁₂-cycloalkyl radical, optionally substituted by one or more C₁-C₄-alkyl groups,
X is an oxygen atom or an NH group,
R₁ is a branched or unbranched C₁-C₁₈-alkyl radical, a C₅-C₁₂-cycloalkyl radical, optionally substituted by one or more C₁-C₄-alkyl groups, or a hydrogen atom, an alkali metal atom, an ammonium group or a group of the formula 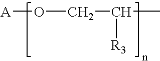
   in which
A is a branched or unbranched C₁-C₁₈-alkyl radical, a C₅-C₁₂-cycloalkyl or aryl radical, optionally substituted by one or more C₁-C₄-alkyl groups,
R₃ is a hydrogen atom or a methyl group,
n is a number of from 1 to 10,
R₂ is a branched or unbranched C₁-C₁₈ alkyl radical, a C₅-C₁₂ cycloalkyl radical, optionally substituted by one or more C₁-C₄ alkyl groups, if X is the NH group, and
   a branched or unbranched C₁-C₁₈ alkyl radical, a C₅-C₁₂ cycloalkyl radical, optionally substituted by one or more C₁-C₄ alkyl groups, or a hydrogen atom, an alkali metal atom, an ammonium group or a group of the formula 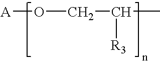
      in which
A is a branched or unbranched C₁-C₁₈ alkyl radical, a C₅-C₁₂ cycloalkyl radical or an aryl radical, optionally substituted by one or more C₁-C₄ alkyl groups,
R₃ is a hydrogen atom or a methyl group,
n is a number of from 1 to 10,
   if X is an oxygen atom.

A particularly preferred UV filter substance for the puposes of the present invention is also an asymmetrically substituted s-triazine, the chemical structure of which is given by the formula 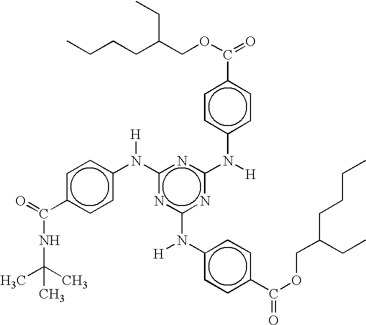
which is also referred to below as dioctylbutylamidotriazone (INCI: Dioctylbutamidotriazone) and is available under the trade name UVASORB HEB from Sigma 3V.

Also advantageous within the scope of the present invention is a symmetrically substituted s-triazine, tris(2-ethylhexyl) 4,4′,4″-(1,3,5-triazine-2,4,6-triyltriimino)trisbenzoate, synonym: 2,4,6-tris [anilino(p-carbo-2′-ethyl-1′-hexyloxy)]-1,3,5-triazine (INCI: Octyl Triazone), which is sold by BASF Aktiengesellschaft under the trade name UVINUL® T 150.

European patent application EP 775 698 also describes advantageously employable bis-resorcinyltriazine derivatives, the chemical structure of which is given by the generic formula 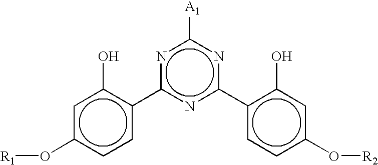
where R₁, R₂ and A₁ represent a large variety of organic radicals.

Also advantageous for the purposes of the present invention are 2,4-bis{[4-(3-sulfonato)-2-hydroxypropyloxy)-2-hydroxy]phenyl}-6-(4-methoxyphenyl)-1,3,5-triazine sodium salt, 2,4-bis{[4-(3-(2-propyloxy)-2-hydroxy]propyloxy)-2-hydroxy]phenyl}-6-(4-methoxyphenyl)-1,3,5-triazine, 2,4-bis-{[4-(2-ethylhexyloxy)-2-hydroxy]phenyl}-6-[4-(2-methoxyethyl-carboxyl)phenylamino]-1,3,5-triazine, 2,4-bis-{[4-(3-(2-propyloxy)-2-hydroxypropyloxy)-2-hydroxy]phenyl}-6-[4-(2-ethylcarboxyl)phenylamino]-1,3,5-triazine, 2,4-bis{[4-(2-ethylhexyloxy)-2-hydroxy]phenyl}-6-(1-methylpyrrol-2-yl)-1,3,5-triazine, 2,4-bis-{[4-tris(trimethylsiloxy-silylpropyloxy)-2-hydroxy]-phenyl}-6-(4-methoxyphenyl)-1,3,5-triazine, 2,4-bis-{[4-(2″-methylpropenyloxy)-2-hydroxy]phenyl}-6-(4-methoxyphenyl)-1,3,5-triazine and 2,4-bis-{[4-(1′,1′,1′,3′,5′,5′,5′-heptamethylsiloxy-2″-methylpropyloxy)-2-hydroxy]phenyl}-6-(4-methoxy-phenyl)-1,3,5-triazine.

An advantageous broadband filter for the purposes of the present invention is 2,2′-methylenebis(6-(2H-benzotriazol-2-yl)-4-(1,1,3,3-tetramethylbutyl)phenol), which is available under the trade name Tinosorb® M from CIBA-Chemikalien GmbH.

Another advantageous broadband filter for the purposes of the present invention is 2-(2H-benzotriazol-2-yl)-4-methyl-6-[2-methyl-3-[1,3,3,3-tetramethyl-1-[(trimethylsilyl)oxy]disiloxanyl]propyl]phenol (CAS No.: 155633-54-8) with the INCI name Drometrizole Trisiloxane.

The UVB and/or broadband filters may be oil-soluble or water-soluble. Examples of advantageous oil-soluble UVB and/or broadband filter substances include:
3-benzylidene camphor derivatives, preferably 3-(4-methylbenzylidene)camphor, 3-benzylidene camphor;
4-aminobenzoic acid derivatives, preferably (2-ethylhexyl) 4-(dimethylamino)benzoate, amyl 4-(dimethylamino)benzoate;
2,4,6-trianilino(p-carbo-2′-ethyl-1′-hexyloxy)-1,3,5-triazine;
esters of benzalmalonic acid, preferably di(2-ethylhexyl) 4-methoxybenzalmalonate;
esters of cinnamic acid, preferably (2-ethylhexyl) 4-methoxycinnamate, isopentyl 4-methoxycinnamate;
derivatives of benzophenone, preferably 2-hydroxy-4-methoxybenzophenone, 2-hydroxy-4-methoxy-4′-methylbenzophenone, 2,2′-dihydroxy-4-methoxy-benzophenone
and UV filters bonded to polymers.

Examples of advantageous water-soluble UVB and/or broadband filter substances include:
salts of 2-phenylbenzimidazole-5-sulfonic acid, such as its sodium, potassium or its triethanolammonium salt, and the sulfonic acid itself;
sulfonic acid derivatives of 3-benzylidene camphor, such as, e.g., 4-(2-oxo-3-bornylidenemethyl)benzenesulfonic acid, 2-methyl-5-(2-oxo-3-bornylidene methyl)-sulfonic acid and salts thereof.

A further light protection filter substance which can be used advantageously according to the invention is ethylhexyl 2-cyano-3,3-diphenylacrylate (octocrylene), which is available from BASF under the designation Uvinul® N 539.

It may also be considerably advantageous to use polymer-bound or polymeric UV filter substances in preparations according to the present invention, in particular those described in WO-A-92/20690.

In addition, it may optionally be advantageous to incorporate further UVA and/or UVB filters into cosmetic or dermatological preparations according to the invention, for example, certain salicylic acid derivatives, such as 4-isopropylbenzyl salicylate, 2-ethylhexyl salicylate (═Octyl salicylate), and homomenthyl salicylate.

Of course, the list of cited UV filters which can be used for the purposes of the present invention is not intended to be limiting.

Preparations according to the invention advantageously contain substances which absorb UV radiation in the UVA and/or UVB range, in a total amount of, e.g., from 0.1% by weight to 30% by weight, preferably from 0.5% to 20% by weight, in particular from 1.0% to 15.0% by weight, based on the total weight of the preparations, in order to make available cosmetic preparations which protect the hair or the skin from the entire range of ultraviolet radiation. They can also be used as sunscreen compositions for the hair or the skin.

The cosmetic and dermatological preparations according to the invention may comprise cosmetic active agents, auxiliaries and additives, as are customarily used in such preparations, e.g., antioxidants, preservatives, bactericides, perfumes, antifoams, dyes, coloring pigments, thickeners, surfactants, emulsifiers, emollients, moisturizers and/or humectants, fats, oils, waxes and other customary constituents of a cosmetic or dermatological formulation, such as alcohols, polyols, polymers, foam stabilizers, electrolytes, organic solvents or silicone derivatives.

If the cosmetic or dermatological preparation according to the present invention is present in the form of a solution or emulsion or dispersion, the following may be used as solvents:
water or aqueous solutions;
oils such as triglycerides of capric or caprylic acid, preferably castor oil;
fats, waxes and other natural and synthetic lipids, preferably esters of fatty acids with alcohols of low C number, for example with isopropanol, propylene glycol or glycerol, or esters of fatty alcohols with alkanoic acids of low C number or with fatty acids;
alcohols, diols or polyols of low C number and their ethers, preferably ethanol, isopropanol, propylene glycol, glycerol, ethylene glycol, ethylene glycol monoethyl ether or monobutyl ether, propylene glycol monomethyl ether, monoethyl ether or monobutyl ether, diethylene glycol monomethyl ether or monoethyl ether, and analogous products.

In particular, mixtures of the above-mentioned solvents may be used. In the case of alcoholic solvents, water may be a further constituent.

The oil phase of the emulsions, oleogels or hydro- or lipodispersions in accordance with the present invention may advantageously be selected from esters of saturated and/or unsaturated, branched and/or unbranched alkanecarboxylic acids with a chain length of from 3 to 30 C atoms and saturated and/or unsaturated, branched and/or unbranched alcohols with a chain length of from 3 to 30 C atoms, from esters of aromatic carboxylic acids and saturated and/or unsaturated, branched and/or unbranched alcohols with a chain length of from 3 to 30 C atoms. In this case, such ester oils may be selected advantageously from isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and synthetic, semisynthetic and natural mixtures of such esters, for example jojoba oil.

Furthermore, the oil phase may advantageously be selected from branched and unbranched hydrocarbons and hydrocarbon waxes, silicone oils, dialkyl ethers, saturated or unsaturated, branched or unbranched alcohols and fatty acid triglycerides, viz. the triglycerol esters of saturated and/or unsaturated, branched and/or unbranched alkanecarboxylic acids with a chain length of from 8 to 24, in particular from 12 to 18, C atoms. For example, the fatty acid triglycerides may advantageously be selected from synthetic, semisynthetic and natural oils, for example olive oil, sunflower oil, soya oil, peanut oil, rapeseed oil, almond oil, palm oil, coconut oil, palm kernel oil and the like.

Any mixtures of such oil and wax components may also advantageously be employed in accordance with the present invention. If appropriate, it may also be advantageous to employ waxes, for example cetyl palmitate, as the only lipid component of the oil phase.

The oil phase may advantageously be selected from 2-ethylhexyl isostearate, octyldodecanol, isotridecyl isononanoate, isoeicosane, 2-ethylhexyl cocoate, C₁₂₋₁₅ alkyl benzoate, caprylic/capric acid triglyceride, dicaprylyl ether.

Especially advantageous mixtures are those of C₁₂₋₁₅ alkyl benzoate and 2-ethylhexyl isostearate, those of C₁₂₋₁₅ alkyl benzoate and isotridecyl isononanoate and those of C₁₂₋₁₅ alkyl benzoate, 2-ethylhexyl isostearate and isotridecyl isononanoate.

Amongst the hydrocarbons, liquid paraffin, squalane and squalene may advantageously be used according to the present invention.

The oil phase may furthermore advantageously comprise cyclic or linear silicone oils, or consist entirely of such oils, but it is preferred to use an additional content of other oil phase components, apart from the silicone oil(s).

Cyclomethicone (octamethylcyclotetrasiloxane) is advantageously employed as silicone oil to be used according to the invention. However, other silicone oils may also be used advantageously in accordance with the present invention, for example, hexamethylcyclotrisiloxane, polydimethylsiloxane, and poly(methylphenylsiloxane).

Especially advantageous mixtures are furthermore those of cyclomethicone and isotridecyl isononanoate and of cyclomethicone and 2-ethylhexyl isostearate.

If appropriate, the aqueous phase of the preparations according to the invention may advantageously comprise alcohols, diols or polyols of low C number, and their ethers, preferably ethanol, isopropanol, propylene glycol, glycerol, ethylene glycol, ethylene glycol monoethyl ether or monobutyl ether, propylene glycol monomethyl ether, monoethyl ether or monobutyl ether, diethylene glycol monomethyl ether or monoethyl ether and analogous products, furthermore alcohols of low C number, for example ethanol, isopropanol, 1,2-propanediol, glycerol, and, in particular, one or more thickeners which may advantageously be selected from silicon dioxide, aluminum silicates, polysaccharides and their derivatives, for example hyaluronic acid, xanthan gum, hydroxypropyl methylcellulose, especially advantageously from polyacrylates, preferably a polyacrylate from the group of the so-called Carbopols, for example type 980, 981, 1382, 2984 and 5984 Carbopols, in each case individually or in combination.

Gels which may be used according to the present invention usually comprise alcohols of low C number, for example ethanol, isopropanol, 1,2-propanediol, glycerol and water, or an above-mentioned oil in the presence of a thickener, which is preferably silicon dioxide or an aluminum silicate in the case of oily-alcoholic gels, and preferably a polyacrylate in the case of aqueous-alcoholic or alcoholic gels.

Solid sticks may comprise, for example, natural or synthetic waxes, fatty alcohols or fatty acid esters.

Customary basic materials which are suitable for use as cosmetic sticks in accordance with the present invention include liquid oils (for example liquid paraffin, castor oil, isopropyl myristate), semi-solid constituents (for example petrolatum, lanolin), solid constituents (for example beeswax, ceresine and micro-crystalline waxes, or ozocerite) and waxes of high melting point (for example carnauba wax and candelilla wax).

Suitable propellants for cosmetic and/or dermatological preparations in accordance with the present invention which can be sprayed from aerosol containers are the customary known volatile, liquefied propellants, for example hydrocarbons (propane, butane, isobutane), which may be employed individually or as a mixture with each other. Pressurized air may also be used advantageously.

Those of skill in the art will, of course, be familiar with the fact that there are non-toxic propellants, which would be suitable in principle for putting into practice the present invention in the form of aerosol preparations; however, it is recommended to dispense with the use of these—in particular fluorohydrocarbons and fluorochlorohydrocarbons (FCHCs)—due to their unacceptable effect on the environment or other accompanying circumstances.

Cosmetic preparations in accordance with the present invention may also take the form of gels which comprise not only an effective amount of active ingredient according to the invention and conventionally used solvents therefor, preferably water, but also organic thickeners, for example gum arabic, xanthan gum, sodium alginate, cellulose derivatives, preferably methylcellulose, hydroxymethylcellulose, hydroxyethylcellulose, hydroxypropylcellulose, hydroxypropyl methylcellulose, or inorganic thickeners, for example, aluminum silicates such as, for example, bentonites, or a mixture of polyethylene glycol and polyethylene glycol stearate or polyethylene glycol distearate. The gel comprises the thickener for example in an amount of between 0.1 and 30% by weight, preferably between 0.5 and 15% by weight.

It is particularly advantageous for the purposes of the present invention if the cosmetic or dermatological preparations according to the present invention contain further active substances, in particular natural active substances and/or derivatives thereof, such as, e.g., alpha-lipoic acid, phytoene, D-biotin, coenzyme Q10, alpha-glucosyl rutin, carnitine, carnosine, osmolytes, clover extract, hop extract or hop-malt extract.

The concentration of the active ingredients (one or more substances) is advantageously from 0.0001% to 30% by weight, based on the total weight of the preparations.

### DETAILED DESCRIPTION OF THE INVENTION

The following examples are to illustrate the present invention. “AFP and/or AFGP”, i.e., anti-freezing proteins and/or anti-freezing glycoproteins are in particular to be understood to mean one or more of the above-mentioned substances.

Unless indicated otherwise, all numbers in the examples are percent by weight, based on the total weight of the preparations.

1. O/W Creams

| | Example | | | | |
|---|---|---|---|---|---|
| | 1 | 2 | 3 | 4 | 5 |
| Glyceryl stearate citrate | | | 2.00 | | 2.00 |
| Glyceryl stearate self-emulsifying | 4.00 | 3.00 | | | |
| PEG-40 stearate | 1.00 | | | | |
| Polyglyceryl-3-methylglucose | | | | 3.00 | |
| distearate | | | | | |
| Sorbitan stearate | | | | | 2.00 |
| Stearic acid | | 1.00 | | | |
| Polyoxyethylene(20) cetyl stearyl | | | | | |
| ether | | | | | |
| Stearyl alcohol | | | 5.00 | | |
| Cetyl alcohol | 3.00 | 2.00 | | 3.00 | |
| Cetyl stearyl alcohol | | | | | 2.00 |
| C₁₂₋₁₅ Alkyl benzoate | | | | | |
| Caprylic/capric triglyceride | 5.00 | 3.00 | 4.00 | 3.00 | 3.00 |
| Octyldodecanol | | | 2.00 | | 2.00 |
| Dicaprylyl ether | | 4.00 | | 2.00 | 1.00 |
| Paraffinum liquidum | 5.00 | 2.00 | | 3.00 | |
| Titanium dioxide | | | 1.00 | | |
| 4-Methylbenzylidene camphor | | | 1.00 | | |
| 1-(4-tert-Butylphenyl)-3-(4- | | | 0.50 | | |
| methoxyphenyl)-1,3-propanedione | | | | | |
| Antifreezing protein of type 1 | 0.20 | 0.50 | 0.10 | 1.00 | 0.30 |
| Tocopherol | 0.1 | | | | 0.20 |
| Biotin | | | 0.05 | | |
| Ethylenediamine tetracarboxylic acid | 0.1 | | 0.10 | 0.1 | |
| trisodium | | | | | |
| Preservative | q.s. | q.s. | q.s. | q.s. | q.s. |
| Xanthan gum | | | | | |
| Polyacrylic acid | 3.00 | 0.1 | | 0.1 | 0.1 |
| Aqueous sodium hydroxide 45% | q.s. | q.s. | q.s. | q.s. | q.s. |
| Glycerin | 5.00 | 3.00 | 4.00 | 3.00 | 3.00 |
| Butylene glycol | | 3.00 | | | |
| Perfume | q.s. | q.s. | q.s. | q.s. | q.s. |
| Water | ad 100 | ad 100 | ad 100 | ad 100 | ad 100 |
| | Example | | | | |
| | 6 | 7 | 8 | 9 | 10 |
| Glyceryl stearate citrate | | 2.00 | 2.00 | | |
| Glyceryl stearate self-emulsifying | 5.00 | | | | |
| Stearic acid | | | | 2.50 | 3.50 |
| Stearyl alcohol | 2.00 | | | | |
| Cetyl alcohol | | | | 3.00 | 4.50 |
| Cetyl stearyl alcohol | | 3.00 | 1.00 | | 0.50 |
| C₁₂₋₁₅ Alkyl benzoate | | 2.00 | 3.00 | | |
| Caprylic/capric triglyceride | 2.00 | | | | |
| Octyldodecanol | 2.00 | 2.00 | | 4.00 | 6.00 |
| Dicaprylyl ether | | | | | |
| Paraffinum liquidum | | 4.00 | 2.00 | | |
| Cyclic dimethylpolysiloxane | | | | 0.50 | 2.00 |
| Dimethicone polydimethylsiloxane | 2.00 | | | | |
| Titanium dioxide | 2.00 | | | | |
| 4-Methylbenzylidene camphor | 1.00 | | | | 1.00 |
| 1-(4-tert-Butylphenyl)-3-(4- | 0.50 | | | | 0.50 |
| methoxyphenyl)-1,3-propanedione | | | | | |
| Antifreezing protein of type 2 | 0.20 | 0.70 | 0.25 | 1.00 | 0.40 |
| Tocopherol | | | | | 0.05 |
| Ethylenediamine tetracarboxylic acid | | | 0.20 | | 0.20 |
| trisodium | | | | | |
| Preservative | q.s. | q.s. | q.s. | q.s. | q.s. |
| Xanthan gum | | | 0.20 | | |
| Polyacrylic acid | 0.15 | 0.1 | | 0.05 | 0.05 |
| Aqueous sodium hydroxide 45% | q.s. | q.s. | q.s. | q.s. | q.s. |
| Glycerin | 3.00 | | 3.00 | 5.00 | 3.00 |
| Butylene glycol | | 3.00 | | | |
| Ethanol | | 3.00 | | 3.00 | |
| Perfume | q.s. | q.s. | q.s. | q.s. | q.s. |
| Water | ad 100 | ad 100 | ad 100 | ad 100 | ad 100 |

2. W/O Emulsions

| | Example | | |
|---|---|---|---|
| | 11 | 12 | 13 |
| Cetyldimethicone copolyol | | 2.50 | |
| Polyglyceryl-2-dipolyhydroxystearate | 5.00 | | 4.50 |
| 2-Ethylhexyl methoxycinnamate | | 8.00 | 4.00 |
| 2,4-Bis-(4-(2-ethylhexyloxy)-2- | 2.00 | 2.50 | 2.50 |
| hydroxy)phenyl-6-(4-methoxyphenyl)- | | | |
| (1,3,5)-triazine | | | |
| Diethylhexyl butamidotriazone | 3.00 | 1.00 | 3.00 |
| Octocrylene | 7.00 | 2.50 | 2.50 |
| Diethylhexyl butamidotriazone | 1.00 | | |
| Phenylene-1,4-bis-(monosodium, 2- | 1.00 | 2.00 | |
| benzimidazyl-5,7-disulfonic acid) | | | |
| Phenylbenzimidazole sulfonic acid | 0.50 | | 2.00 |
| Titanium dioxide | | 2.00 | 3.00 |
| Zinc oxide | 3.00 | 1.00 | |
| Paraffinum liquidum | | | 8.00 |
| Dicaprylyl ether | 10.00 | | 7.00 |
| Butylene glycol dicaprylate/dicaprate | | | 4.00 |
| Dicaprylyl carbonate | 5.00 | | |
| Dimethicone polydimethylsiloxane | | 4.00 | |
| Phenyl methyl polysiloxane | 2.00 | 25.00 | 2.00 |
| PVP hexadecene copolymer | 0.50 | | 1.00 |
| Octoxyglycerin | | 0.30 | 0.50 |
| Glycerin | 3.00 | 7.50 | 2.50 |
| Glycine soya | | 1.00 | |
| Magnesium sulfate | 1.00 | 0.50 | |
| Magnesium chloride | | | 0.70 |
| Antifreezing protein of type 3 | 0.10 | 0.60 | 0.80 |
| Preservative | q.s. | q.s. | q.s. |
| Ethanol | 3.00 | | 1.00 |
| Perfume | q.s. | q.s. | q.s. |
| Water | ad 100 | ad 100 | ad 100 |

| | Example | |
|---|---|---|
| | 14 | 15 |
| Cetyldimethicone copolyol | | 4.00 |
| PEG-30 dipolyhydroxy stearate | 5.00 | |
| 2-Ethylhexyl methoxycinnamate | | 5.00 |
| 2,4-Bis-(4-(2-ethylhexyloxy)-2-hydroxy)phenyl- | | 2.00 |
| 6-(4-methoxyphenyl)-(1,3,5)-triazine | | |
| 1-(4-tert-Butylphenyl)-3-(4-methoxyphenyl)-1,3- | 2.00 | 1.00 |
| propanedione | | |
| Ethylhexyl triazone | 3.00 | 4.00 |
| Octocrylene | 4.00 | |
| Diethylhexyl butamidotriazone | | 2.00 |
| Phenylene-1,4-bis-(monosodium, 2-benzimidazyl- | 0.50 | |
| 5,7-disulfonic acid) | | |
| Phenylbenzimidazole sulfonic acid | | 3.00 |
| Titanium dioxide | 1.50 | |
| Zinc oxide | 2.00 | 0.50 |
| Paraffinum liquidum | 10.0 | |
| C₁₂₋₁₅ Alkyl benzoate | | 9.00 |
| Butylene glycol dicaprylate/dicaprate | 2.00 | 8.00 |
| Dicaprylyl carbonate | 6.00 | |
| Dimethicone polydimethylsiloxane | 1.00 | 5.00 |
| PVP hexadecene copolymer | | 0.50 |
| Octoxyglycerin | 1.00 | |
| Glycerin | | 7.50 |
| Glycine soya | 1.50 | |
| Magnesium sulfate | 0.70 | 0.50 |
| Antifreezing protein of type 1 | 1.00 | 1.00 |
| Preservative | q.s. | q.s. |
| Ethanol | 1.50 | |
| Perfume | q.s. | q.s. |
| Water | ad 100 | ad 100 |
| | Example | |
| | 16 | 17 |
| Polyglyceryl-2-dipolyhydroxystearate | 4.00 | 5.00 |
| PEG-30 dipolyhydroxystearate | | |
| Lanolin alcohol | 0.50 | 1.50 |
| Isohexadecane | 1.00 | 2.00 |
| Myristyl myristate | 0.50 | 1.50 |
| Vaseline | 1.00 | 2.00 |
| 1-(4-tert-Butylphenyl)-3-(4-methoxyphenyl)-1,3- | 0.50 | 1.50 |
| propanedione | | |
| 4-Methylbenzylidene camphor | 1.00 | 3.00 |
| Butylene glycol dicaprylate/dicaprate | 4.00 | 5.00 |
| Shea butter | | 0.50 |
| Butylene glycol | | 6.00 |
| Octoxyglycerin | | 3.00 |
| Glycerin | 5.00 | |
| Tocopherol acetate | 0.50 | 1.00 |
| Antifreezing protein of type 1 | 0.20 | 0.25 |
| Trisodium EDTA | 0.20 | 0.20 |
| Preservative | q.s. | q.s. |
| Ethanol | | 3.00 |
| Perfume | q.s. | q.s. |
| Water | ad 100 | ad 100 |

3. Hydrodispersions

| | Example | |
|---|---|---|
| | 18 | 19 |
| Polyoxyethylene(20) cetyl stearyl ether | 1.00 | |
| Sodium polyacrylate | | 0.20 |
| Acrylate/C10-30 Alkylacrylate Crosspolymer | 0.50 | |
| Xanthan gum | | 0.30 |
| 2,4-Bis-(4-(2-ethylhexyloxy)-2-hydroxy)phenyl-6-(4- | | 1.50 |
| methoxyphenyl)-(1,3,5)-triazine | | |
| 1-(4-tert-Butylphenyl)-3-(4-methoxyphenyl)-1,3- | 1.00 | |
| propanedione | | |
| Diethylhexyl butamidotriazone | | 2.00 |
| Ethylhexyl triazone | 4.00 | |
| Octocrylene | | 4.00 |
| Phenylene-1,4-bis-(monosodium, 2-benzimidazyl- | 1.00 | |
| 5,7-disulfonic acid) | | |
| Phenylbenzimidazole sulfonic acid | 0.50 | |
| Titanium dioxide | 0.50 | |
| Zinc oxide | 0.50 | 1.00 |
| C₁₂₋₁₅ Alkyl benzoate | 2.00 | 2.50 |
| Dicaprylyl ether | | 4.00 |
| Butylene glycol dicaprylate/dicaprate | 4.00 | |
| Dicaprylyl carbonate | | 2.00 |
| Dimethicone polydimethylsiloxane | | 0.50 |
| Phenyl methyl polysiloxane | 2.00 | |
| Shea butter | | 2.00 |
| PVP hexadecene copolymer | 0.50 | |
| Glycerin | 3.00 | 7.50 |
| Tocopherol acetate | 0.50 | |
| Antifreezing glycoprotein | 0.15 | 0.60 |
| Preservative | q.s. | q.s. |
| Ethanol | 3.00 | 2.00 |
| Perfume | q.s. | q.s. |
| Water | ad 100 | ad 100 |

| | Example 20 |
|---|---|
| Cetyl alcohol | 1.00 |
| Acrylate/C10-30 Alkylacrylate Crosspolymer | 0.40 |
| Xanthan gum | 0.15 |
| 1-(4-tert-Butylphenyl)-3-(4-methoxyphenyl)-1,3- | 2.00 |
| propanedione | |
| Ethylhexyl triazone | 3.00 |
| Octocrylene | 4.00 |
| Phenylene-1,4-bis-(monosodium, 2-benzimidazyl-5,7- | 0.50 |
| disulfonic acid) | |
| Titanium dioxide | 2.00 |
| Zinc oxide | 3.00 |
| Butylene glycol dicaprylate/dicaprate | 2.00 |
| Dicaprylyl carbonate | 6.00 |
| Dimethicone polydimethylsiloxane | 1.00 |
| Octoxyglycerin | 1.00 |
| Glycine soya | 1.50 |
| Tocopherol acetate | 0.25 |
| Antifreezing glycoprotein | 1.00 |
| Preservative | q.s. |
| Ethanol | 1.50 |
| Perfume | q.s. |
| Water | ad 100 |

| | Example | |
|---|---|---|
| | 21 | 22 |
| Polyoxyethylene(20) cetyl stearyl ether | 0.5 | |
| Sodium polyacrylate | 0.30 | |
| Acrylate/C10-30 Alkylacrylate Crosspolymer | 0.10 | 0.10 |
| Xanthan gum | | 0.50 |
| 2-Ethylhexyl methoxycinnamate | 5.00 | 8.00 |
| 2,4-Bis-(4-(2-ethylhexyloxy)-2-hydroxy)phenyl-6-(4- | 2.00 | 2.50 |
| methoxyphenyl)-(1,3,5)-triazine | | |
| Diethylhexyl butamidotriazone | 2.00 | 1.00 |
| Ethylhexyl triazone | 4.00 | |
| 4-Methylbenzylidene camphor | | 2.00 |
| Octocrylene | | 2.50 |
| Phenylene-1,4-bis-(monosodium, 2-benzimidazyl- | | 2.00 |
| 5,7-disulfonic acid) | | |
| Phenylbenzimidazole sulfonic acid | 3.00 | |
| Titanium dioxide | 3.00 | 1.00 |
| Zinc oxide | | 2.00 |
| Butylene glycol dicaprylate/dicaprate | 6.00 | |
| Phenyl methyl polysiloxane | 0.50 | 2.00 |
| PVP hexadecene copolymer | 0.50 | 1.00 |
| Octoxyglycerin | | 0.50 |
| Glycerin | 7.50 | 2.50 |
| Tocopherol acetate | | 1.00 |
| Antifreezing protein of type 2 | 1.00 | 0.80 |
| Preservative | q.s. | q.s. |
| Ethanol | | 1.00 |
| Perfume | q.s. | q.s. |
| Water | ad 100 | ad 100 |

4. Gel Cream

| | Example 23 |
|---|---|
| Acrylate/C10-30 Alkylacrylate Crosspolymer | 0.40 |
| Polyacrylic acid | 0.20 |
| Xanthan gum | 0.10 |
| Cetearyl alcohol | 3.00 |
| C₁₂₋₁₅ Alkyl benzoate | 4.00 |
| Caprylic/capric triglyceride | 3.00 |
| Cyclic dimethylpolysiloxane | 5.00 |
| Dimethicone polydimethylsiloxane | 1.00 |
| Antifreezing protein of type 3 | 0.20 |
| Glycerin | 3.00 |
| Sodium hydroxide | q.s. |
| Preservative | q.s. |
| Perfume | q.s. |
| Water | ad 100.0 |

pH adjusted to 6.0

5. W/O Cream

| | Example 24 |
|---|---|
| Polyglyceryl-3-diisostearate | 3.50 |
| Glycerin | 3.00 |
| Polyglyceryl-2-dipolyhydroxy stearate | 3.50 |
| Antiofreezing protein of type 3 | 0.50 |
| Preservative | q.s. |
| Perfume | q.s. |
| Water | ad 100.0 |
| Magnesium sulfate | 0.6 |
| Isopropyl stearate | 2.0 |
| Caprylyl ether | 8.0 |
| Cetearyl isononanoate | 6.0 |

6. W/O/W Cream

| | Example 25 |
|---|---|
| Glyceryl stearate | 3.00 |
| PEG-100 stearate | 0.75 |
| Behenyl alcohol | 2.00 |
| Caprylic/capric triglyceride | 8.0 |
| Octyldodecanol | 5.00 |
| C₁₂₋₁₅ Alkyl benzoate | 3.00 |
| Antifreezing protein of type 1 | 1.00 |
| Magnesium sulfate (MgSO₄) | 0.80 |
| Ethylenediamine tetracarboxylic acid | 0.10 |
| Preservative | q.s. |
| Perfume | q.s |
| Water | ad 100.0 |

pH adjusted to 6.0

7. W/O Sticks

| | Example 26 |
|---|---|
| PEG-45/dodecyl glycol copolymer | 2.00 |
| Polyglyceryl-3-diisostearate | 2.00 |
| Caprylic/capric triglyceride | 4.00 |
| Cetearyl isononanoate | 4.00 |
| Butylene glycol dicaprylate/dicaprate | 5.00 |
| Ethylhexyl methoxycinnamate | 5.00 |
| Ethylhexyl triazone | 3.00 |
| Bis-ethylhexyloxyphenol methoxyphenyl triazine | 2.50 |
| Hombitec H | 2.00 |
| C₂₀₋₄₀ Alkyl stearate | 9.00 |
| Silica dimethylsilylate | 1.00 |
| Dimethicone | 0.50 |
| Glycerin | 10.00 |
| Antifreezing glycoprotein | 0.20 |
| PVP hexadecene copolymer | 0.50 |
| Tocopherol acetate | 1.00 |
| Preservative | q.s. |
| Perfume | q.s. |
| Water | ad 100 |
| | Example 27 |
| PEG-45/dodecyl glycol copolymer | 2.00 |
| Polyglyceryl-3-diisostearate | 2.00 |
| Cetearyl isononanoate | 15.00 |
| C₂₀₋₄₀ Alkyl stearate | 8.00 |
| Glycerin | 10.00 |
| Antifreezing protein of type 4 | 0.50 |
| Preservative | q.s. |
| Perfume | q.s. |
| Water | ad 100.00 |

8. O Stick

| | Example 28 |
|---|---|
| Microcrystalline wax | 25.00 |
| Carnauba wax | 2.00 |
| Cetearyl alcohol | 2.20 |
| Octyldodecanol | 14.00 |
| Ethylhexyl methoxycinnamate | 3.20 |
| 1-(4-tert-Butylphenyl)-3-(4-methoxyphenyl)-1,3- | 0.40 |
| propanedione | |
| 4-Methylbenzylidene camphor | 1.50 |
| Cetyl palmitate | 15.00 |
| Shea butter | 1.00 |
| Paraffinum liquidum | 17.00 |
| Castor oil | 12.00 |
| Titanium dioxide | 5.00 |
| Tocopherol acetate | 1.00 |
| Perfume | q.s. |
| Antifreezing protein of type 2 | 0.20 |

9. W/O Cream

| | Example 29 |
|---|---|
| Polyglyceryl-2-dipolyhydroxystearate | 4.50 |
| Polyglyceryl-3-diisostearate | 0.50 |
| Lanolin alcohol | 1.00 |
| Vaseline | 4.00 |
| Caprylic/capric triglyceride | 2.00 |
| Titanium dioxide | 2.00 |
| Ethylhexyl methoxycinnamate | 5.00 |
| Ethylhexyl triazone | 3.00 |
| Bis-ethylhexyloxyphenol methoxyphenyl triazine | 2.50 |
| Isopropyl stearate | 2.0 |
| Caprylyl ether | 8.0 |
| Cetearyl isononanoate | 6.0 |
| Glycerin | 6.00 |
| Magnesium sulfate | 0.60 |
| Tocopherol acetate | 1.00 |
| Ethylenediamine tetracarboxylic acid | 0.10 |
| Preservative | q.s. |
| Perfume | q.s. |
| Water | ad 100.0 |
| Antifreezing protein of type 4 | 0.20 |

10. O Cream

| | Example 30 |
|---|---|
| Vaseline | 43.50 |
| Paraffinum liquidum | 33.00 |
| Paraffinum wax | 2.50 |
| Microcrystalline wax | 1.50 |
| Lanolin alcohol | 0.50 |
| Talc | 4.00 |
| Zinc oxide | 4.00 |
| Octyldodecanol | 3.00 |
| Cyclomethicone | 2.50 |
| Antifreezing protein of type 3 | 0.20 |
| Perfume | q.s. |

What is claimed is:

1. A cosmetic or dermatological preparation comprising one or more proteins which are selected from anti-freezing proteins and anti-freezing glycoproteins.

2. The preparation of claim 1, wherein the preparation comprises at least one anti-freezing protein.

3. The preparation of claim 1, wherein the preparation comprises at least one anti-freezing glycoprotein.

4. The preparation of claim 1, wherein the one or more proteins are present in a concentration of from 0.0001% to 50% by weight, based on a total weight of the preparation.

5. The preparation of claim 4, wherein the one or more proteins are present in a concentration of at least 0.001% by weight.

6. The preparation of claim 4, wherein the one or more proteins are present in a concentration of from 0.1% to 10% by weight.

7. The preparation of claim 6, wherein the one or more proteins are present in a concentration of up to 1% by weight.

8. The preparation of claim 2, wherein the at least one anti-freezing protein comprises at least one protein selected from types AFP 1, AFP 2, AFP 3 and AFP 4.

9. The preparation of claim 2, wherein the at least one anti-freezing protein comprises at least one protein of type AFP 1 that is synthesized by at least one of pseudopluronectes americanus, myoxocephalus scorpius, myoxocephalus aenaeus and myoxocephalus scorpiodes.

10. The preparation of claim 2, wherein the at least one anti-freezing protein comprises at least one protein of type AFP 2 that is synthesized by at least one of hemitripterus americanus, osmerus mordax and clupea harengus harengus.

11. The preparation of claim 2, wherein the at least one anti-freezing protein comprises at least one protein of type AFP 3 that is synthesized by at least one of macrozoarces americanus, rhigophila dearbomi lycodes polaris and the “wolffish” anarhichas lupus.

12. The preparation of claim 2, wherein the at least one anti-freezing protein comprises at least one protein of type AFP 4 that is synthesized by myoxocephalus octodecimspinosis.

13. The preparation of claim 3, wherein the at least one anti-freezing glycoprotein comprises at least one protein that is synthesized by at least one of trematomas borgrevinki, dissostichus mawsoni, boreogadus saida and gadus morhua.

14. A cosmetic or dermatological preparation comprising one or more proteins which are selected from anti-freezing proteins and anti-freezing glycoproteins that are synthesized by at least one of pseudopluronectes americanus, myoxocephalus scorpius, myoxocephalus aenaeus, myoxocephalus scorpiodes, hemitripterus americanus, osmerus mordax, clupea harengus harengus, macrozoarces americanus, rhigophila dearbomi, lycodes polaris, anarhichas lupus, myoxocephalus octodecimspinosis, trematomas borgrevinki, dissostichus mawsoni, boreogadus saida and gadus morhua.

15. The preparation of claim 14, wherein the one or more proteins are present in a concentration of from 0.001% to 50% by weight, based on a total weight of the preparation.

16. The preparation of claim 15, wherein the one or more proteins are present in a concentration of from 0.1% to 10% by weight.

17. The preparation of claim 1, wherein at least a part of the one or more proteins is encapsulated.

18. A cosmetic or dermatological product comprising the preparation of claim 1, wherein the product is selected from o/w creams, w/o creams, w/o/w creams, o creams, w/o emulsions, hydrodispersions, gel creams, w/o sticks and o sticks.

19. A method for the treatment or prevention of undesirable conditions of skin, wherein the method comprises applying to at least parts of the skin one or more proteins selected from anti-freezing proteins and anti-freezing glycoproteins.

20. The method of claim 19, wherein the undesirable skin conditions include at least one of skin inflammation, pigmentation disorders, symptoms of extrinsic and intrinsic skin aging, and skin damage caused by UV radiation.
